(19) United States
(12) Patent Application Publication
(10) Pub. No.: US 2009/0159779 A1
(43) Pub. Date: Jun. 25, 2009

(54) LIGHT CURING APPARATUS

(75) Inventor: YU-HAN LU, Tu-Cheng,Taipei Hsien (TW)

Correspondence Address: PCE INDUSTRY, INC.;ATT. Steven Reiss, 458 E. LAMBERT ROAD, FULLERTON, CA, 92835, US

(73) Assignee: HON HAI PRECISION INDUSTRY CO., LTD., Tu-Cheng,Taipei Hsien (TW)

(21) Appl. No.: 12/202,545

(22) Filed: Sep. 2, 2008

(30) Foreign Application Priority Data
Dec. 24, 2007 (CN) 200710203363.5

(51) Int. Cl. G01J 1/32; A61N 5/06

(52) U.S. Cl. 250/205; 250/504 R

(57) ABSTRACT

A light curing apparatus including a carrier device and a light source with a lamp. The light curing apparatus also includes a light intensity detection device, a data processing unit and a display unit. The light intensity detection device is configured for detecting the intensity of the light source and outputting a light intensity value, the data processing unit is configured for receiving the light intensity value and forming and outputting a data signal of the changed curve of light intensity with the time change, the display unit is configured for receiving the output of the data processing unit and displaying the curve of the light intensity with the time change. The light curing apparatus can detect the actual light intensity of the light source, such that the light curing time can be changed accordingly.

### BACKGROUND

1. Related Art

The present invention relates to a light curing apparatus and, particularly, to a light curing apparatus providing adjustable curing parameters.

2. Description of Related Art

Light-cured adhesives are utilized in a wide variety of manufacturing process. An example is a typical camera module, which includes at least one lens and at least one spacer. During assembly of the camera module, the lens and the spacer are combined using light-cured adhesive, and the combination thereof is sent to a curing apparatus. Curing parameters of the apparatus are commonly set based on the specification of the curing apparatus, e.g., light-intensity of a lamp thereof. As the lamp ages, the light-intensity thereof. The curing parameters of the apparatus may not meet the desired standard as a result, which can adversely affect the quality of adhesive bonding.

It is thus desired to provide a light curing apparatus providing adjustable curing parameters, which can overcome the described limitations.

### SUMMARY

In accordance with the present invention, a light curing apparatus solidifying adhesive on a workpiece includes a carrier device configured for carrying the workpiece, a light source, a light-intensity detection device, a display device, and an adjustment device. The light source is configured for generating light, which solidifies the adhesive on the workpiece. The light-intensity detection device is configured for detecting an intensity of the light from the light source and generating an intensity value of the light. The display device is configured for displaying the intensity value. The adjustment device is configured for adjusting the irradiation time for the workpiece and light intensity of the light source according to the intensity value

Other novel features and advantages will become more apparent from the following detailed description when taken in conjunction with the accompanying drawings.

### BRIEF DESCRIPTION OF THE DRAWINGS

The present invention is described in detail hereinafter, by way of example and description of preferred and exemplary embodiments thereof with reference to the accompanying drawings, in which:

FIG. 1 is a schematic view of a curing apparatus according to a first exemplary embodiment;

FIG. 2 is a flowchart of a method for curing adhesive using the curing apparatus of FIG. 1.

### DETAILED DESCRIPTION OF THE EXEMPLARY EMBODIMENTS

A detailed explanation of a light curing apparatus according to an exemplary embodiment will now be made with reference to the drawings attached hereto.

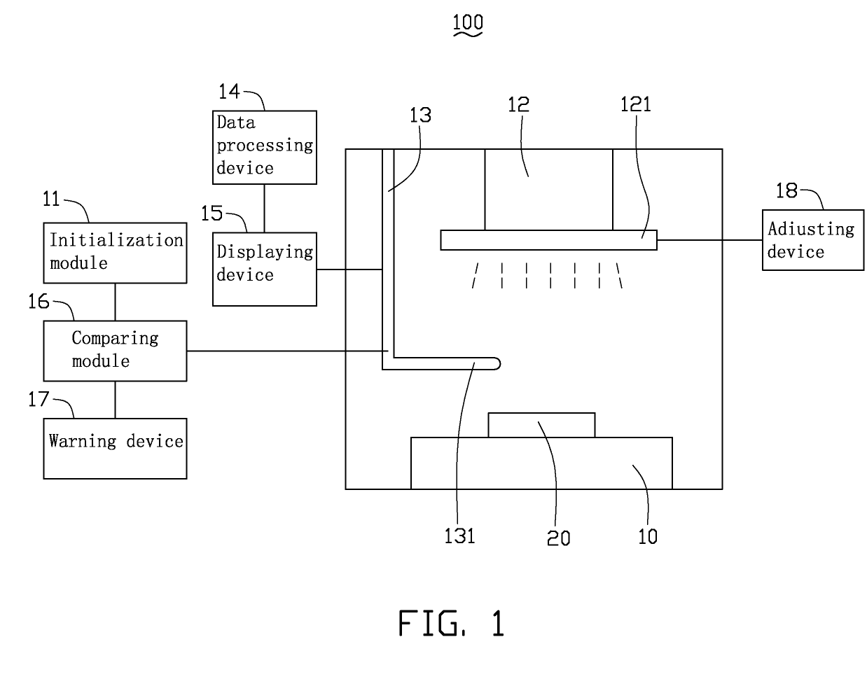

Referring to FIG. 1, a light curing apparatus 100 according to an exemplary embodiment is shown. The light curing apparatus 100 includes a carrier device 10, an initialization module 11, a light source 12, a light-intensity detection device 13, a data processing device 14, a display device 15, a comparison module 16, a warning device 17, and an adjustment device 18.

The carrier device 10 is configured for carrying a workpiece 20 and may be a static or movable platform. The movable platform may be connected to a driver mechanism (not shown) such that the platform can be moved along the xy-plane to selectively position the workpiece 20. Understandably, the carrier device 10 also may be a conveyer belt for conveying the workpiece 20. In the present embodiment, the carrier device 10 is a static platform.

The initialization module 11 is configured for initializing a light-intensity standard value of the light source 12. The light-intensity standard value is employed in comparison with the actual intensity value of the light source 12 to determine the light source condition.

The light source 12 includes a lamp 121 and is configured for generating light towards the adhesive and curing it. The lamp 121 may be an ultraviolet lamp that uses ultraviolet light emitting diode (LED) as light source.

The light-intensity detection device 13 is electronically connected to the light source 12 and configured for detecting an intensity of the light generated from the light source 12 and generating an intensity value thereof. When the lamp 121 of the light source 12 is an ultraviolet LED, the light-intensity detection device 13 will be an ultraviolet sensor. The light-intensity detection device 13 includes a detection end 131 between the carrier device 10 and the lamp 121, exposed to light emitted from the light source 12. Such that, the light-intensity detection device 13 can receive the light and obtain the intensity value of the light according to the intensity thereof.

The data processing device 14 is electronically connected to the light-intensity detection device 13 and configured for processing the intensity value and plotting a graph according to the intensity value. The graph represents a relationship between the intensity value and the serve time of the light source 12.

The display device 15 is electronically connected to the data processing device 14 and configured for displaying the graph and the intensity value.

The comparison module 16 is electronically connected to the initialization module 11 and the light-intensity detection device 13, and configured for comparing the intensity value with the light-intensity standard value. Intensity value exceeding the light-intensity standard value indicates that source 12 is in good condition. When the intensity value is less than the light-intensity standard value, the intensity of the light source 12 requires adjustment.

The warning device 17 is electronically connected to the comparison module 16 and configured for sending a warning signal when the intensity value falls below the light-intensity standard value. The warning device 17 may be LEDs of standard or other colors. In this embodiment, the warning device 17 is a red LED.

The adjustment device 18 is electronically connected to the light source 12 and configured for adjusting the light intensity of the light source 12 and the irradiation time for each workpiece according to the intensity value obtained by the light-intensity detection device 13. As will be apparent to those skilled in the art, the adjustment of the light intensity and the irradiation time for the light source 12 are achieved via changes made to the voltage or current supplied to the light source 12.

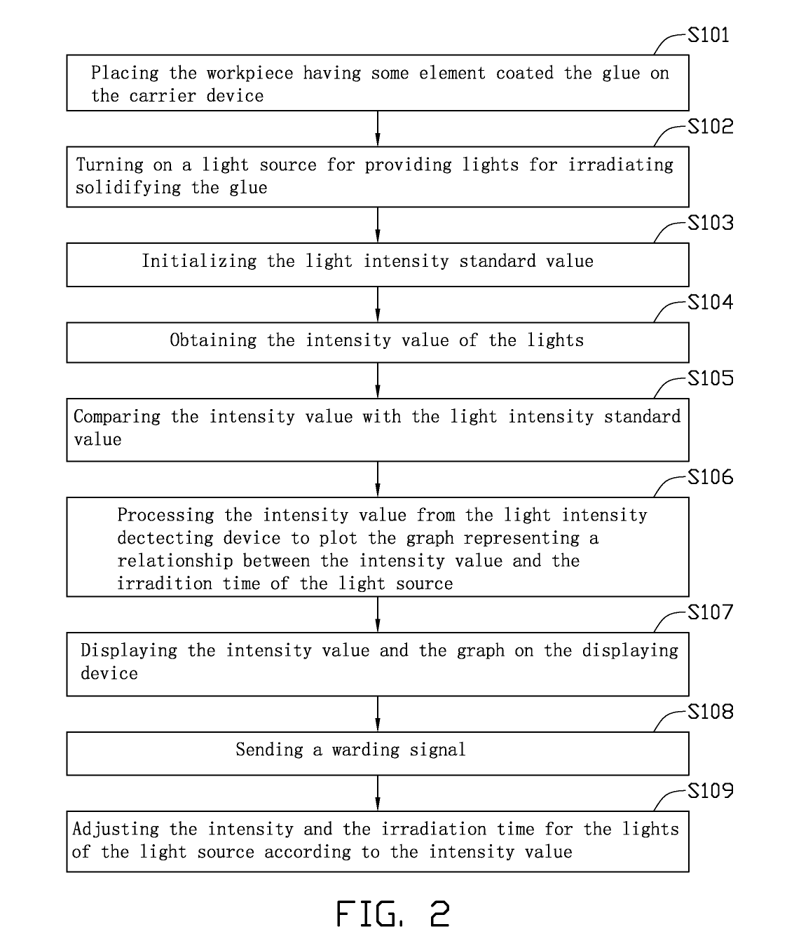

Referring to FIG. 2, as shown in a flowchart of an exemplary method for curing the adhesive of the workpiece is shown. The method includes step S101, in which the workpiece, including the adhesive is placed on the carrier device 10. In step S102, light source 12 is activated, irradiating and curing the adhesive. In step S103, the light intensity standard value is initialized. In step S104, the intensity value of light generated from the light source 12 is obtained. In step S105, the obtained intensity value is compared with the light intensity standard value. In step S106, the intensity value from the light intensity detection device is processed to plot a graph representing a relationship between the intensity value and the irradiation time of the light source. In step S107, the intensity value and graph are shown on the display device 15, and in step S108, a warning signal is issued. In step S109, intensity and the irradiation time for the light of the light source 12 are adjusted according to the intensity value.

As described, the user can adjust the light intensity and exposure time of the light source 12 using the adjustment device 18. Allowing adhesive of the workpiece to be cured effectively. Moreover, lamp 121 of the light source 12 can be replaced according to the light-intensity value, which can avoid inefficient functioning of the light curing apparatus 100.

It should be understood that the above-described embodiment is intended to illustrate rather than limit the invention. Variations may be made to the embodiments without departing from the spirit of the invention. Accordingly, it is appropriate that the appended claims be construed broadly and in a manner consistent with the scope of the invention.

What is claimed is:

1. A light curing apparatus for curing a light curable adhesive applied to a combination of elements, the apparatus comprising:
a carrier device configured for carrying the combination;
a light source configured for generating light which, upon irradiating the adhesive, cures the adhesive, fixing the adhesion of the combination;
a light-intensity detection device configured for detecting intensity of light from the light source and generating an intensity value thereof;
a display device configured for displaying the generated intensity value;
an adjustment device configured for adjusting exposure time of irradiation of the combination and intensity of the light according to the intensity value.

2. The light curing apparatus as claimed in claim 1, wherein the light source comprises a lamp.

3. The light curing apparatus as claimed in claim 2, wherein the lamp is an ultraviolet lamp and the light-intensity detection device comprises an ultraviolet sensor.

4. The light curing apparatus as claimed in claim 2, wherein the lamp comprises a light emitting diode.

5. The light curing apparatus as claimed in claim 1, comprising a data processing device, configured for processing the intensity value from the light intensity detection device and plotting a graph showing a relationship between the intensity value and the service time of the light source.

6. The light curing apparatus as claimed in claim 5, wherein the data processing device is a central processer.

7. The light curing apparatus as claimed in claim 1, further comprising an initialization module configured for initializing a light-intensity standard value, a comparison module configured for comparing the light-intensity standard value with the intensity value, and a warning device issuing a warning signal when the current received intensity value is less than the light-intensity standard value.

8. The light curing apparatus as claimed in claim 7, wherein the warning device comprises a red light emitting diode.

9. The light curing apparatus as claimed in claim 1, wherein the display device comprises a touch screen.

10. A method for curing a light curable adhesive applied to a combination of elements, the method comprising:
placing the combination on a carrier device;
activating a light source generating light, which, when irradiating the adhesive, cures the adhesive;
generating an intensity value of the generated light;
displaying the intensity value on a display device;
adjusting the intensity of light of the light source according to the intensity value.

11. The method as claimed in claim 10, further comprising:
initializing a light-intensity standard value before exposure of the adhesive;
comparing the intensity value with the light-intensity standard value after obtaining the intensity value; and
issuing a warning signal when the intensity value is less than the light-intensity standard value.

12. The method as claimed in claim 11, wherein the warning signal is sent by a warning device.

13. The method as claimed in claim 12, wherein the warning device comprises a red light emitting diode.

14. The method as claimed in claim 12, wherein the warning device comprises a yellow light emitting diode.

15. The method as claimed in claim 10, further comprising:
processing the intensity value from the light intensity detection device to form a curve after obtaining the intensity value; and
plotting a graph, the graph representing a relationship between the intensity value and the service time of the light source.
